(19) United States
(12) Patent Application Publication
(10) Pub. No.: US 2021/0299299 A1
(43) Pub. Date: Sep. 30, 2021

(54) DEVICE FOR ERADICATING BACTERIA AND VIRUSES

(71) Applicant: Ray SC LLC, West Palm Beach, FL (US)

(72) Inventor: Salvatore Furia, Rydal, PA (US)

(21) Appl. No.: 17/214,107

(22) Filed: Mar. 26, 2021

Related U.S. Application Data
(60) Provisional application No. 63/000,242, filed on Mar. 26, 2020.
(60) Provisional application No. 63/013,157, filed on Apr. 21, 2020.

(51) Int. Cl. A61L 2/10; A61L 2/24

(52) U.S. Cl. CPC A61L 2/10; A61L 2/24; A61L 2202/16; A61L 2202/11; A61L 2202/14

(57) ABSTRACT

A device for eradicating bacteria and viruses using ultraviolet C (UV-C) light. The device includes a two-dimensional array of ultraviolet (UV) light sources and a two-dimensional array of sensors interspersed between the UV light sources. A structural skeleton may surround the UV light sources and the sensors. The structural skeleton may be configured to support the weight of a user wearing shoes such that the user can stand on the device above the planes created by the UV light array and the sensor array. Critically, the device includes a controller that determines the location of an object on a top surface of the device based on the output of the sensors and controls the UV light sources to emit UV-C light towards the bottom surface of the object.

### CROSS REFERENCE TO RELATED APPLICATIONS

This application claims priority to U.S. Prov. Pat. Appl. No. 63/000,242, filed Mar. 26, 2020, and U.S. Prov. Pat. Appl. No. 63/013,157, filed Apr. 21, 2020, which are hereby incorporated by reference.

### BACKGROUND

Ultraviolet C (UV-C) light is highly effective at killing bacteria and viruses by destroying the molecular bonds that hold their DNA together. Broad-spectrum germicidal UV-C light, which has a wavelength between 224 and 285 nanometers (nm), is particularly effective. Accordingly, conventional UV-C light is routinely used to decontaminate surgical equipment.

Unfortunately, conventional germicidal UV-C light may also be a human health hazard that can lead to skin cancer and cataracts. Therefore, there is a need for a device that uses UV-C light to kill bacteria and viruses on objects, in particular human footwear, while minimizing the human wearer's exposure to UV-C light.

### SUMMARY

In order to overcome those and other drawbacks in the prior art, a device is provided for eradicating bacteria and viruses using ultraviolet C (UV-C) light. The device includes a two-dimensional array of ultraviolet (UV) light sources and a two-dimensional array of sensors interspersed between the UV light sources. A structural skeleton may surround the UV light sources and the sensors. The structural skeleton may be configured to support the weight of a user wearing shoes such that the user can stand on the device above the planes created by the UV light array and the sensor array. Critically, the device includes a controller that determines the location of an object on a top surface of the device based on the output of the sensors and controls the UV light sources to emit UV-C light towards the bottom surface of the object.

### BRIEF DESCRIPTION OF THE DRAWINGS

The accompanying drawings are incorporated in and constitute a part of this specification. Features in the accompanying drawings are illustrated for clarity and are not necessary drawn to scale. It is to be understood that the drawings illustrate only some examples of the disclosure and other examples or combinations of various examples that are not specifically illustrated in the figures may still fall within the scope of this disclosure. Examples will now be described with additional detail through the use of the drawings.

FIG. 1 is a top-down view of a mat-like device for eradicating bacteria and viruses using ultraviolet C (UV-C) light according to an exemplary embodiment.

FIG. 2 is an exploded view of the interior components of the device according to an exemplary embodiment, which include a sensor array and a UV light array,

FIG. 3 is another view of the sensor array and the UV light array according to an exemplary embodiment.

FIG. 4 is a side view of the interior components of the device according to an exemplary embodiment.

### DETAILED DESCRIPTION

In describing the illustrative, non-limiting embodiments illustrated in the drawings, specific terminology will be resorted to for the sake of clarity. However, the disclosure is not intended to be limited to the specific terms so selected, and it is to be understood that each specific term includes all technical equivalents that operate in similar manner to accomplish a similar purpose. Several embodiments are described for illustrative purposes, it being understood that the description and claims are not limited to the illustrated embodiments and other embodiments not specifically shown in the drawings may also be within the scope of this disclosure.

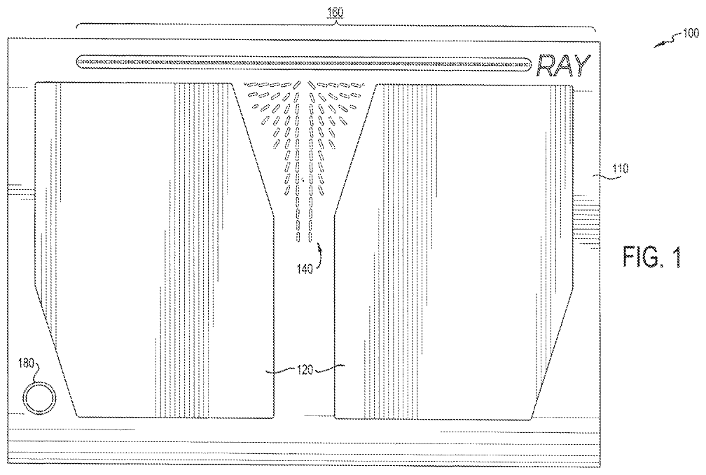

FIG. 1 is a top-down view of a mat-like device 100 for eradicating bacteria and viruses using ultraviolet C (UV-C) light according to an exemplary embodiment.

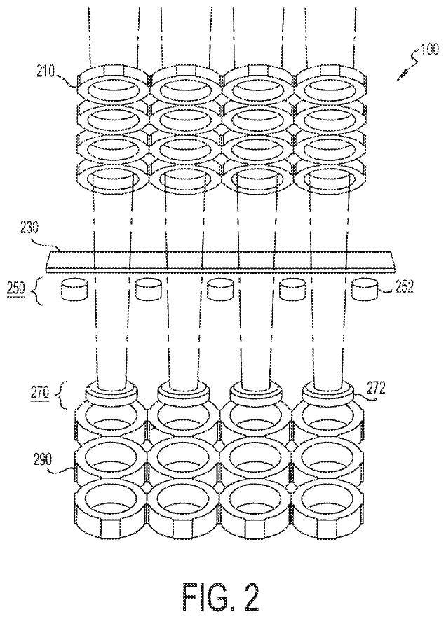

FIG. 2 is an exploded view of the interior components of the device 100 according to an exemplary embodiment.

As shown in FIG. 2, the device 100 may include a skeletal base 290, a UV light array 270, a sensor array 250, a protective film 230, and a skeletal cap 210.

The UV light array 270 may be a two-dimensional array of UV light sources 272 that emit UV-C light in a direction substantially orthogonal to the plane created by the UV light array 270. The UV light sources 272 may be light emitting diodes (LEDs). Alternatively, the UV light sources 272 may be one or more UV tube lamps and computer-controlled windows (similar to a liquid crystal display). The UV light sources 272 may emit UV-C light having a wavelength between 224 and 285 nanometers (nm). More specifically, the UV light sources 272 may emit UV-C light having a wavelength between 265 and 285 nm.

The skeletal base 290 and the skeletal cap 210 may be configured to support the weight of a user wearing shoes such that the user can stand on the device 100 above the plane created by the UV light array 270. The UV light sources 272 can then emit UV-C light upward toward the bottom surface of the user's shoes, thereby eradicating an overwhelming majority (e.g., 99 percent or 99.9 percent) of the bacteria and viruses on the bottom surface of the user's shoes.

Since UV-C light can be harmful to humans, the device 100 is configured to emit light from the UV light sources 272 that are below an object (e.g., a user's shoes) while minimizing the UV-C light emitted from the UV light sources 272 that are not below an object. To do so, the sensor array 250 may include a two-dimensional array of sensors 252. Each sensor 252 is configured to determine if an object (e.g., a user's shoe) is on the device above the location of the sensor 252. Each sensor 252 may be a proximity sensor (e.g., an LED sensor), a force sensor, a switch, etc. The sensor array 250 creates a plane that is substantially parallel to the plane of the UV light array 270.

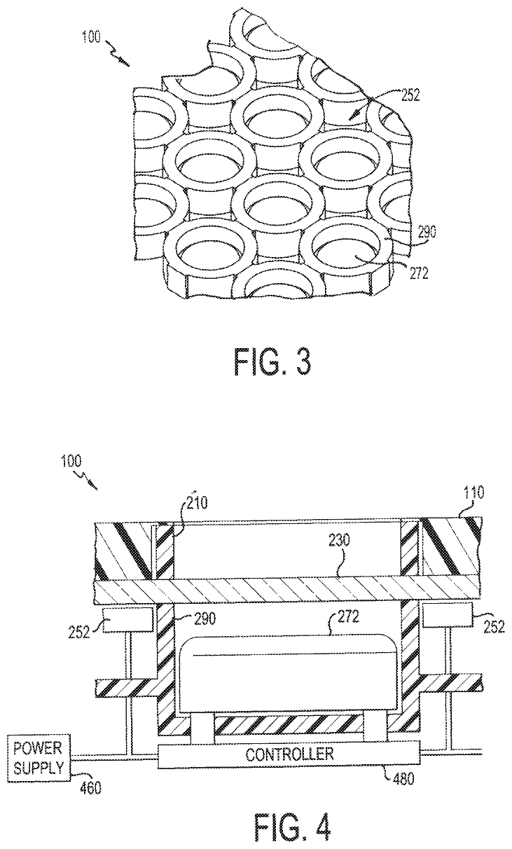

FIG. 3 is another view of the sensor array 250 and the UV light array 270 according to an exemplary embodiment.

Looking from the top-down (in the direction orthogonal to the planes of the sensor array 250 and the UV light array 270), the sensor array 250 overlaps with the UV light array 270 such that the sensors 252 are interspersed between the UV light sources 272. For example, each sensor 252 may be surrounded by four UV light sources 272 and each UV light source 272 may be surrounded by four sensors 252.

FIG. 4 is a side view of the interior components of the device 100 according to an exemplary embodiment.

As shown in FIG. 4, the device 100 includes a power supply 460 as well as a controller 480 electrically connected to each of the UV light sources 272 and each of the sensors 252. The controller 480 receives the output of each of the sensors 252 and controls the UV light sources 272 based on the output of the sensors 252. The controller 480 determines the location of one or more objects on the device 100 (based on the output of the sensors 252) and causes the UV light sources 272 below the objects to emit UV-C light towards the objects.

In some embodiments, the controller 480 may cause one UV light source 272 to emit UV-C light in response to multiple sensors 252. Alternatively, the controller 480 may cause multiple UV light sources 272 to emit UV-C light in response to one sensor 252. For example, each sensor 252 may be surrounded by a certain number of UV light sources 272 (e.g., four UV light sources 272) and the controller 480 may turn on the UV light sources 272 that are adjacent to each sensor 252 that sense an object above the sensor 252. As a result, all of the UV light sources 272 below an object will emit UV-C light toward the underside of the object. Meanwhile, the UV light sources 272 that are not covered by an object will not emit UV-C light (with the possible exception of a few UV light sources 272 immediately adjacent to an object covering an adjacent sensor 252). Turning on the UV light sources 272 that are under objects (as opposed to the entire UV light array 270) minimizes the potentially harmful UV-C light transmitted to the user's skin or eyes and extends the life span of the UV light sources 272.

The controller 480 may be any suitable device capable of performing the functions described herein. The controller 480 may be an electronic circuit, such as a hardware processor.

To protect the UV light sources 272 and the sensors 252, the device may also include a skeletal base 290 that is configured to support the weight of a user. As shown in FIGS. 2-4, the skeletal base 290 may surround each of the UV light sources 272 and each of the sensors 252. The top side of the skeletal base 290 includes openings that enable the UV light sources 272 to emit UV-C light in the direction of an object on the device 100. If the sensors 252 are proximity sensors, the skeletal base 290 may also include openings that allow the proximity sensors 252 to determine if an object is on top of the device 100 in the location above the proximity sensor 252. The device may also include a skeletal cap 210 with substantially the same shape as the skeletal base 290. The device may also include a protective film 230 between the skeletal cap 210 and the skeletal base 290 that protects the UV light sources 272 and/or sensors 252. The protective film 230 is transparent to the UV-C light. If the sensors 252 are LED sensors, the protective film 230 is transparent to light in the wavelength of the LED sensors 252.

The power supply 460 may include a battery and/or a wired connection to a power source. The battery may be rechargeable. The device 100 may be portable, in which case the power supply 460 may include a battery. The device 100 may be integrated into a floor, in which case the power supply 460 may include a persistent wired connection to a power source.

Referring back to FIG. 1, the device 100 may include a top cover 110 with two sensor-activated foot panels 120, a path guide 140, a progress bar 160, and a battery level indicator 180.

Each foot panel 120 may be situated above a UV light array 270 and a sensor array 250 as described above. In some embodiments, the device 100 is configured to output UV-C light for a predetermined time period that has been determined to be sufficient to eradicate a sufficient amount (e.g., 99 percent or 99.9 percent) of the bacteria and viruses on the bottom surface of a user's shoes. In those embodiments, users are expected to stand in place on the foot panels 120 for the predetermined time period. In those embodiments, the device 100 may include an output device that outputs an indication that the eradication process has begun. The output device may output a visual indication, an audible indication, a tactile indication, etc. For example, the path guide 140 may include lights that output a visual indication that the eradication process has begun. In those embodiments, the device 100 may further include an output device that outputs an indication that the predetermined time period is ongoing and/or that the predetermined time period is complete. The output device may output a visual indication, an audible indication, a tactile indication, etc. For example, the progress bar 160 may be a line of LED lights that output a visual indication of the UV emission process and/or an indication that the predetermined time period is complete. In another embodiment, the output device may be a speaker that outputs an audible indication of the UV emission process (e.g., a song that plays until the process is complete) and/or an audible indication that the predetermined time period is complete (e.g., a sound indicating that the process is complete).

In other embodiments, the UV light array 270 and sensor array 250 may be relatively long (e.g., along a walkway). In those embodiments, users are expected to walk across the device 100 as usual while the device 100 eradicates the bacteria and viruses on the bottom surface of the user's shoes as the user is in motion. In those embodiments, the device 100 (e.g., walkway) may be sufficiently long enough that the shoes of a typical user walking across the device will receive sufficient UV-C light to eradicate a sufficient amount (e.g., 99 percent or 99.9 percent) of the bacteria and viruses on the bottom surface of the user's shoes. In those embodiments, each UV light source 272 may emit UV-C light only in response to a determination that a user's shoe is above the UV light source 272 (e.g., in response to a determination from an adjacent sensor 252 that an object is above the adjacent sensor 252). In case a user stands still, the device may be configured such that each UV light source 272 turns off after the predetermined time period even in response to a determination that an object is above the UV light source 272 for longer than the predetermined time period. This configuration saves power and extends the lifespan of the UV light source 272 while preventing unnecessary exposure. Alternatively, the UV light sources 272 may emit UV-C light having a wavelength (e.g., 222 nm) where continuous or long duration illumination is permissible.

As briefly mentioned above, the power supply 460 may include a battery. In those embodiments, the device 100 may include an output device that outputs an indication of the charge level of the battery. The output device may output a visual indication, an audible indication, a tactile indication, etc. For example, the battery level indicator 180 may include lights that output a visual indication indicative of the charge level of the battery. For instance, the battery level indicator 180 may change color when the charge level of the battery is below a predetermined threshold. Alternatively, the battery level indicator 180 may include a number of lights that are activated and deactivated proportional to the charge level of the battery.

The top cover 110 of the device may be detachable. As such, the device 100 may include multiple interchangeable covers 110 with unique designs that allow the user to change the ornamental design of the top of the device 100. The top cover 110 of the device may be transparent or translucent. The transparent or translucent top cover 110 may not be detachable.

In some embodiments, the device 100 may be configured to output information to an external device. For example, the device 100 may communicate output information to a user device (e.g., a smartphone, a tablet, a personal computer, etc.), for example using a short-range wireless communications protocol (e.g., Bluetooth), or via a local area network (using a wired or wireless connection). In another example, the device 100 may output information to a server (e.g., via the local area network and the internet), which may provide the information to user devices (e.g., via a smartphone application, a website, etc.). In yet another example, multiple devices 100 may communicate with each other (e.g., via a wired or wireless network connection).

The device 100 may output information indicative of the battery charge, a notification that the battery needs to be recharged, information indicative of the lifespan of the UV light sources 272, a notification that one or more of the UV light sources 272 are approaching the end of their estimated life span, etc. To encourage users to get the benefits provided by the device 100, the device 100 may also include additional features. For example, the device may determine and track a user's weight and output that information to the user device.

The foregoing description and drawings should be considered as illustrative only of the principles of the disclosure, which may be configured in a variety of shapes and sizes and is not intended to be limited by the embodiment herein described. Numerous applications of the disclosure will readily occur to those skilled in the art. Therefore, it is not desired to limit the disclosure to the specific examples disclosed or the exact construction and operation shown and described. Rather, all suitable modifications and equivalents may be resorted to, falling within the scope of the disclosure.

1. A device for eradicating bacteria and viruses using ultraviolet C (UV-C) light, the device comprising:
a two-dimensional array of ultraviolet (UV) light sources;
a two-dimensional array of sensors, the sensors being interspersed between the UV light sources;
a structural skeleton that surrounds the UV light sources and the sensors;
a controller in electrical communication that:
determines the location of an object on a top surface of the device based on the output of the sensors; and
controls the UV light sources to emit UV-C light towards the bottom surface of the object.

2. The device of claim 1, wherein:
each sensor includes one or more UV light sources adjacent to the sensor;
the controller determines the location of the object and causes the UV light sources to emit UV-C light towards the bottom surface of the object by causing the adjacent UV light sources to emit UV-C light in response to a determination that the sensor detects an object above the sensor.

3. The device of claim 1, wherein the UV light sources are light emitting diodes (LEDs).

4. The device of claim 1, wherein the UV light sources emit light having a wavelength between 224 and 285 nanometers.

5. The device of claim 1, wherein the UV light sources emit light having a wavelength between 265 and 285 nanometers.

6. The device of claim 1, wherein the sensors are proximity sensors.

7. The device of claim 6, wherein the proximity sensors are LED sensors.

8. The device of claim 1, wherein the sensors are force sensors or switches.

9. The device of claim 1, wherein the device is portable.

10. The device of claim 1, wherein the device is integrated into a floor.

11. A method of eradicating bacteria and viruses using ultraviolet C (UV-C) light by a device that includes a two-dimensional array of ultraviolet (UV) light sources, a two-dimensional array of sensors, and a structural skeleton that surrounds the UV light sources and the sensors, the method comprising:
receiving, by a controller, the output of the sensors;
determining, by the controller, the location of an object above the device based on the output of the sensors; and
emitting UV-C light, by the UV light sources, towards the bottom surface of the object.

12. The method of claim 11, wherein:
each sensor includes one or more UV light sources adjacent to the sensor;
the controller determines the location of the object and causes the UV light sources to emit UV light towards the bottom surface of the object by causing the adjacent UV light sources to emit UV light in response to a determination that the sensor detects an object above the sensor.

13. The method of claim 11, wherein the UV light sources are light emitting diodes (LEDs).

14. The method of claim 11, wherein the UV light sources emit light having a wavelength between 200 and 400 nanometers.

15. The method of claim 11, wherein the UV light sources emit light having a wavelength between 265 and 285 nanometers.

16. The method of claim 11, wherein the sensors are proximity sensors.

17. The method of claim 16, wherein the proximity sensors are LED sensors.

18. The method of claim 11, wherein the sensors are force sensors or switches.

19. The method of claim 11, wherein the device is portable.

20. The method of claim 11, wherein the device is integrated into a floor.
